(19) United States
(12) Patent Application Publication
(10) Pub. No.: US 2005/0154348 A1
(43) Pub. Date: Jul. 14, 2005

(54) Breast pump

(76) Inventors: Daniel Lantz, Morristown, NJ (US); Anthony Pigliacampo, Menlo Park, CA (US); Michael Strasser, San Francisco, CA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/831,895

(22) Filed: Apr. 26, 2004

Related U.S. Application Data
(60) Provisional application No. 60/535,469, filed on Jan. 8, 2004.

(51) Int. Cl. A61M 1/06

(52) U.S. Cl. 604/74

(57) ABSTRACT

A breast pump, for example a manual or mechanical breast pump, that provides massaging to the areola and/or milk lake region of the breast sequentially or simultaneously. The breast pump, for example, features an actuator that provides both positive and negative pressure during a single stroke. Flexible membranes within the funnel of the breast pump inflate to massage the breast, especially the nipple and milk lake regions.

### FIELD OF THE INVENTION

The present invention relates to a breast pump and in particular to a manually operated breast pump which has a massaging funnel to facilitate and/or enhance the expression of milk during breast pumping.

### BACKGROUND OF THE INVENTION

Breast pumps provide a means for a nursing mother to collect breast milk and/or to relieve discomfort from breast engorgement when the mother is away from her infant, for example when she is away from home at work. Despite the existence of breast pumps for years, many currently marketed breast pumps are sub-optimal because they are not efficacious, not comfortable or both.

Research in the filed of human lactation has shown that stimulation of the breast is an important factor in milk production. Positive pressure on various regions of the breast results in a massaging effect that stimulates the breast. For example, U.S. Pat. No. 6,273,868 and U.S. patent application Ser. No. 10/220,034, both of which are hereby incorporated by reference, discuss two particular regions of the breast that are manipulated to enhance milk expression.

The first region, for example, is the “milk lake region” of the breast, which as defined herein, refers to the edges of the areola, which is situated approximately 2.5 to 3 cm from the base of the nipple. Not wishing to be bound to any particular scientific theory, it is believed that positive pressure on this milk lake region causes the pituitary gland to release oxytocin which is a hormone that causes the milk ducts to dilate in order to be emptied.

The second region of stimulation, for example, is the nipple itself. As with the milk lake region, positive pressure and/or manipulation of the nipple causes release of hormones that enhance milk expression.

Positive pressure on these two particular regions in combination with a negative pressure placed on the breast after release of or simultaneously with the positive pressure causes the breast to be massaged and to let down milk.

Many of the currently marketed breast pumps incorporate an intermittent negative pressure in the funnel to withdraw breast milk. Few pumps, mostly electric breast pumps, utilize any positive pressure on the breast. Of those pumps that do incorporate positive pressure, they do not actively place positive pressure on the areola and/or milk lake region of the breast. Thus, there is a need for a breast pump, for example a manual breast pump, that stimulates milk production through the generation of positive pressure on both the areola and milk lake region either simultaneously or sequentially.

Another disadvantage with breast pumps, especially manual breast pumps, is that they have been relatively complicated and difficult to disassemble for cleaning and difficult to assemble for use. For example, some manual breast pumps have as many as nine separate parts to clean and sanitize. Thus, there is a need for a manual breast pump that includes a few number of components that require assembly prior to use.

The breast pump of the present invention features a breast pump funnel that massages the breast through the use of positive pressure. The breast pump also withdraws milk from the milk ducts through the use of negative pressure. Although the breast pump of the present invention contains multiple components, the components are grouped together, for example through pre-assembly, such that the user does not have many parts to sanitize or assemble prior to use.

### SUMMARY OF THE INVENTION

The present invention provides a breast pump, for example a manual or mechanical breast pump, that massages the breast when being pumped. The massage is implemented by placing at least one discrete area of positive pressure on the breast. Moreover, a massaging wave, described in more detail below, can be created on the breast by the breast pump of the present invention. For example, at least two discrete areas of positive pressure on the breast, for example, on the areola and milk lake region, are implemented sequentially or simultaneously during a single stroke of a pressure generating mechanism, for example, a piston or a diaphragm.

These and other features, advantages and objects of the present invention will be further understood and appreciated by those skilled in the art by references to the following specification, claims and appended drawings.

### BRIEF DESCRIPTION OF THE DRAWINGS

The accompanying drawings, which are incorporated in and constitute a part of the specification, illustrate an exemplary embodiment of the present invention.

FIG. 1 shows a perspective view of a manual breast pump in accordance with an exemplary embodiment of the present invention;

FIG. 2 shows a side elevational view of a housing for a manual breast pump in accordance with an exemplary embodiment of the present invention;

FIG. 3 shows a side elevational view of an actuator connected to a handle in accordance with an exemplary embodiment of the present invention;

FIG. 4 shows an exploded perspective view of an actuator and a handle in accordance with an exemplary embodiment of the present invention;

FIG. 5 shows a perspective view of the underside of a handle in accordance with an exemplary embodiment of the present invention;

FIG. 6 shows a side elevational view of a housing, an inner funnel and a handle in accordance with an exemplary embodiment of the present invention;

FIG. 7 shows a side elevational view of the funnel portion in accordance with an exemplary embodiment of the present invention;

FIG. 8 shows a front perspective view of the funnel portion in accordance with an exemplary embodiment of the present invention;

FIG. 9 shows a side cross-sectional view of the breast pump with the handle in the neutral position in accordance with an exemplary embodiment of the present invention;

FIG. 10 shows a side cross-sectional view of the breast pump with then handle in a position between the neutral position and the engaged position in accordance with an exemplary embodiment of the present invention;

FIG. 11 shows a side cross-sectional view of the breast pump with the handle in the engaged position in accordance with an exemplary embodiment of the present invention;

FIG. 12 shows an enlargement of the inset shown in FIG. 11;

FIG. 13 shows a side cross-sectional view of the breast pump with the handle in a partially engaged position in accordance with an exemplary embodiment of the present invention;

FIG. 14 shows a top plan view of a breast pump with the handle in alignment, i.e. 180°, with respect to the funnel portion in accordance with an exemplary embodiment of the present invention; and

FIG. 15 shows a top plan view of a breast pump with the handle rotated 45° counterclockwise with respect to the funnel portion in accordance with an exemplary embodiment of the present invention.

### DETAILED DESCRIPTION OF THE INVENTION

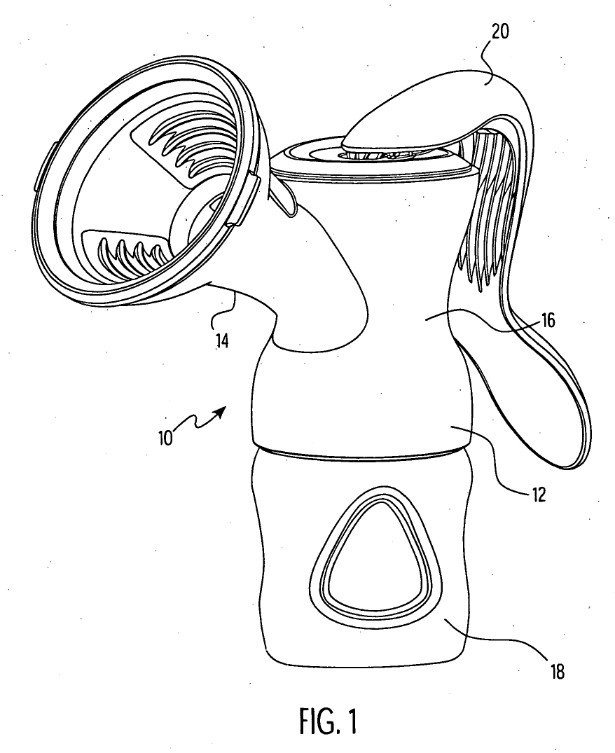

As used herein, the term “proximal” refers to a location with respect to the device during normal use or the element being described that is situated or nearest the base of the breast of a mother when using the present invention. Conversely, the term “distal” refers to a location with respect to the device during normal use or the element being described that is furthest from the base of the breast. As used herein, the term “top”, “up” or “upwardly” refers to the same direction that is relatively orthogonally away from the surface which the breast pump in FIG. 1 sits upon. Conversely, as used herein, the term “bottom”, “down” or “downwardly” refers to the same direction that is relatively orthogonally towards the surface which the breast pump sits upon. As used herein, the term “in”, “inner” or “inwardly” refers to a direction towards the inside of the breast pump funnel portion. Conversely, as used herein, the term “out”, “outer” or “outwardly” refers to a direction towards the outside of the breast pump funnel portion. As used herein, the terms “positive pressure” and “negative pressure” are relative terms. A negative pressure, for example, could mean that the pressure is less positive than another pressure. Nevertheless, negative pressure is commonly less than ambient pressure, for example a vacuum.

The present invention features a breast pump capable of massaging a woman's breast during milk expression. FIG. 1 shows a perspective view of an exemplary manual or mechanical breast pump 10 of the present invention. The breast pump 10 includes a housing 12 that has a funnel portion 14 and a body 16. Along the inner surface of the bottom of the body 16 are, for example, threads (not shown) that can be releasably screwed onto a collection bottle 18, or other container that can receive the breast milk that is expressed from the mother. Extending outwardly from the top of the body 16 is a handle 20 used by the mother for pumping. The breast pump 10 is configured to stably rest on a support surface, for example a table, or rest upon a bottle stand that is then placed on top of a support surface.

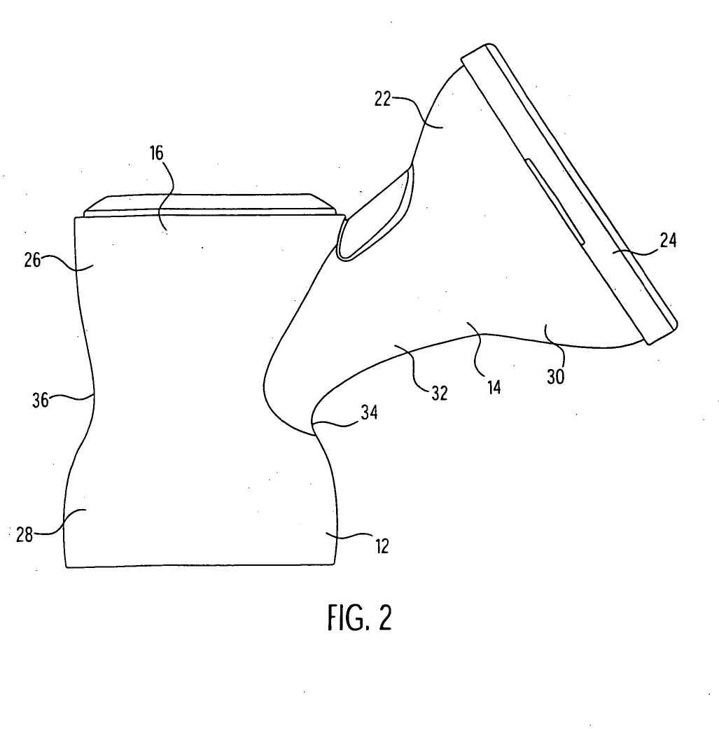

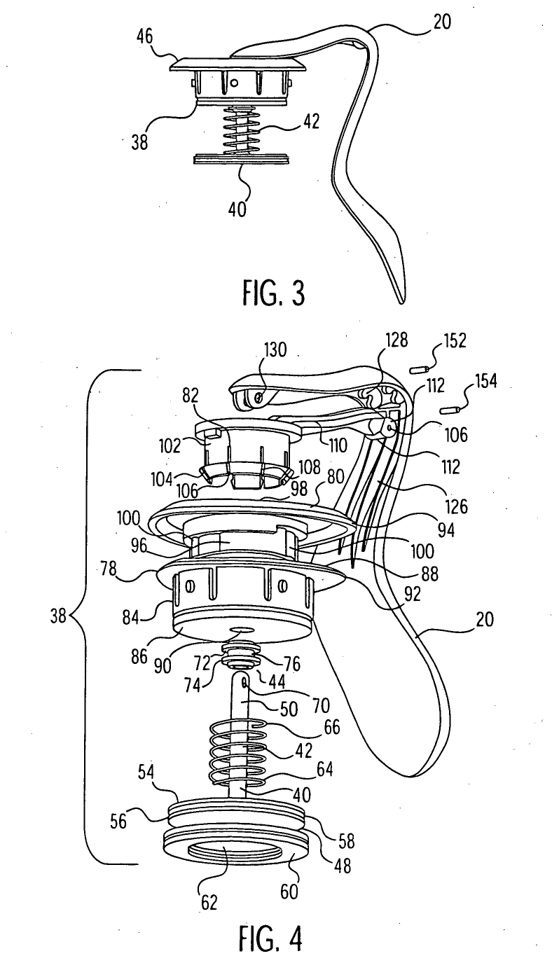

FIG. 2 is a side view of the housing 12. The body 16 of the housing 12 has an hourglass shape that defines an upper compartment 26 and a lower compartment 28. The upper compartment 26 holds the actuator 38 (shown in FIGS. 3 and 4) that provides the positive pressure and negative pressure that are transferred to the funnel portion 14. The funnel portion 14 comprises an outer funnel 22 and an inner funnel 24. The outer funnel 22 has a conical section 30 that is dimensioned to fit a woman's breast. For example, the portion of the breast that includes the nipple and milk-lake region comfortably fits within the conical section 30. The conical section 30 tapers into and connects to a generally cylindrical section 32 of the funnel portion 14. Seated within the outer funnel 22 is the inner funnel 24. The inner funnel 24, described in more detail below, sealingly engages and massages the breast during use.

Where the cylindrical section 32 of the outer funnel 22 meets the outer surface of the lower compartment 28 is a first grip 34, for example, a concave arcuate contour of the housing 12. On the opposite side, for example directly opposite, is a second grip 36 which is also, for example a concave arcuate contour of the housing.12. When in use, the user's hand wraps around the outer surface of the lower compartment 28 and/or collection bottle 18 with her thumb and index finger fitting into the first grip 34 and second grip 36 respectively or vice versa.

The funnel portion 14 and body 16 can be made as an integral part or as separate components that are subsequently assembled together, for example by snap-fit or friction-fit, by the user.

FIGS. 3 and 4 shows side view of an assembled actuator 38 with handle 20 and a perspective view of an exploded actuator 38 and handle 20. The actuator 38, for example, comprises a piston 40, a spring 42, a grommet seal 44, and a lid 46. The piston 40 comprises a circular disc 48 that has a rod 50 extending perpendicularly from the top surface of the disc 48. Around the top and bottom perimeters of the disc 48 are a top flange 54 and a bottom flange 56 respectively. The top flange 54 and bottom flange 56 define a groove 58 therebetween. A ring-shaped piston seal 60 has a ribbed surface along its inner circumference and defines a seal opening 62 of seal 60. The seal 60, for example, is made of a resilient polymer, for example a thermoplastic elastomer. The disc 48 is inserted into the seal opening 62 of the seal 60 such that the groove 58 and ribbed surface of seal 60 form an fluid-tight closure. Alternatively, the seal 60 can be overmolded around the circumference of the disc 48.

As shown in the present embodiment, the mechanism for generating the positive and/or negative pressure is a piston 40. One of ordinary skill in the art can substitute other pressure generating mechanisms for the piston 40, for example a diaphragm or a bellow.

A spring 42, for example a coil spring, has a sufficient diameter such that the rod 50 can be movably positioned within the coils of the spring 42. The spring 42 is removably mounted on the rod 50 such the first coil 64 abuts the top flange 54. It is contemplated that the spring 42 can be a separate, replaceable component. A pinhole 70 runs through the end of the rod 50 opposite the disc 48. Adjacent to the last coil 66 of the spring 42 is the grommet seal 44. The grommet seal 44 has a hole therethrough such that the end of the rod 50 fits through the grommet seal 44. Extending radially from the outer surface of the grommet seal 44 and positioned inwardly from the respective ends of the grommet seal 44 are a top ring 72 and a bottom ring 74 that define a conduit 76 therebetween.

The lid 46 comprises a lid underside 78, a lid base 80 and a rotating center 82. The lid 46 forms the top of the actuator 38 and seals off the upper compartment 26 of the housing 12, as described in greater detail below. The lid underside 78 is cup-shaped having a side wall 84 and a bottom wall 86 that together define a central compartment 88. Centrally located and extending through the bottom wall is a receiving hole 90. The receiving hole 90 is approximately dimensioned such that the grommet seal 44 can be releasably inserted therein. Around the perimeter of the top of the lid underside 78 is a first lip 92. The perimeter of the first lip 92 should have approximately the same size and shape as the top perimeter of the upper compartment 26 of the housing 12. The lid base 80 includes a second lip 94 that is approximately dimensioned and shaped as the first lip 92. Extending from the bottom surface of the second lip 94 is an extending wall 96. The second lip 94 together with the extending wall 96 define a central opening 98. Along the inner surface of the extending wall 96 are multiple position ribs 100 protruding inwardly. The position ribs 100 are located at specific angular distances from each other; for example at thirty degree intervals or at forty-five degree intervals. When the lid base 80 is nested within the lid underside 78, the first lip 92 abuts on the second lip 94, and the side wall 84 is adjacent to the extending wall 96. Furthermore, the central compartment 88 is in communication with the central opening 98. Although as explained above, the lid underside 78 and the lid base 80 are separate components; however, the lid underside 78 and the lid base 80 can be integrally formed or joined after formation, for example by ultrasonic welding.

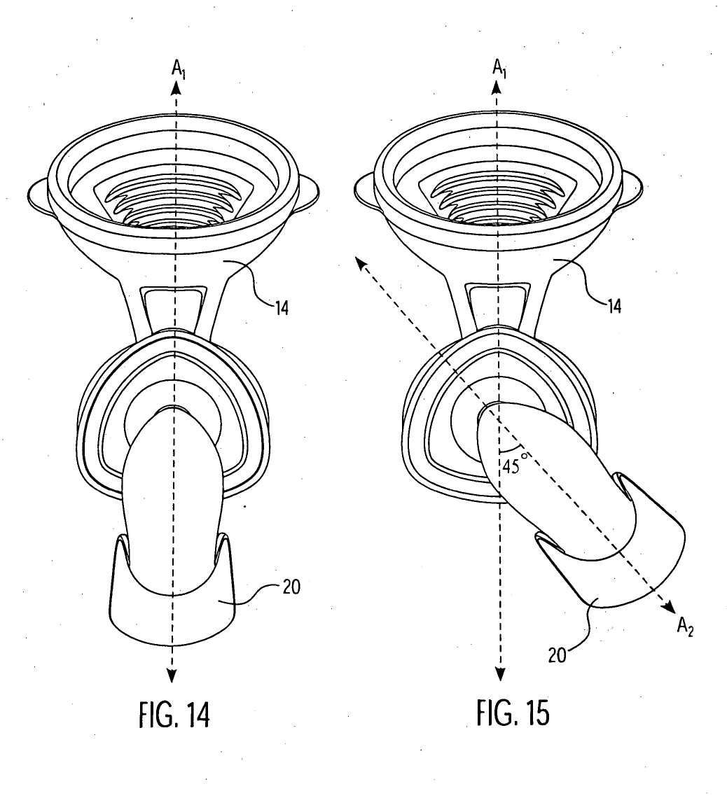

The rotating center 82 connects the handle 20 to the actuator 38. Furthermore, the rotating center 82 allows the handle 20 to be axially rotated with respect to the housing 12 and funnel portion 14. This rotating feature allows the user to find a more comfortable pump orientation to operate the breast pump 10 when the breast pump 10 contacts her breasts. FIG. 14 shows the handle 20 in alignment (as shown by the dotted arrow A₁), i.e. 180°, with respect to the funnel portion 14. FIG. 15 shows the handle 20 rotated, for example, by 45° counterclockwise with respect to the funnel portion 14 (as shown by the dotted arrow A₂). The rotating center 82 comprises a shaft 102 that has at its bottom surface a locking flange 104. The shaft 102 also defines a central bore 106 extending in the axial direction therethrough. Located around the longitudinal perimeter of the shaft 102 are cut-outs 108. The cut-outs 108 are numbered and dimensioned such that they correspond with the multiple position ribs 100. The multiple position ribs 100 along with the cut-outs 108 form a detent. Extending upwardly and radially from the top circumference of the shaft 102 is a handle support 110 The handle support 110 is in a fixed position relative to the shaft 102. Extending through the width of the handle support 110 at the end opposite the bore 106 is a support bar 112 that defines a support receiving bore 106. When the rotating center 82 is inserted into the central opening 94 of the lid base 80, the locking flange 104 engages the bottom perimeter of the extending wall 96. This locking mechanism prevents the rotating center 82 from being accidentally disengaged from the lid base 80; however, the locking mechanism does not cause the rotating center 82 to be fixed with respect to the lid base 80. The rotating center 82 is still rotatable within the lid base 80 at the specific positions and intervals defined by the multiple position ribs 100.

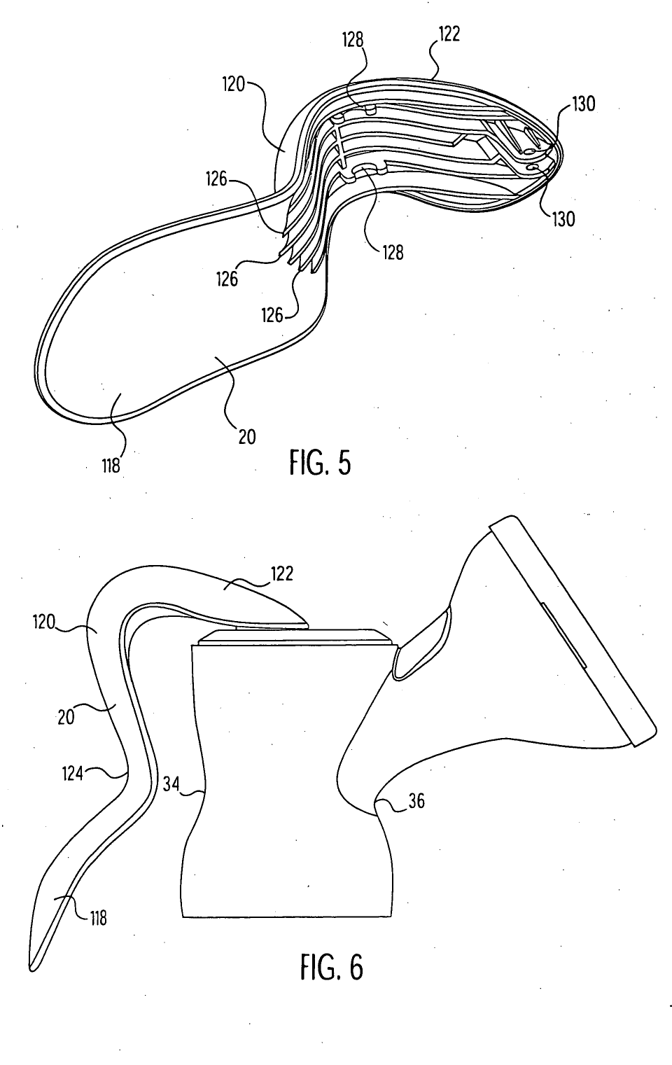

Referring to FIGS. 3, 5 and 6, the handle 20 is connected to the actuator 38. When viewing the handle 20 from the side, as in FIG. 6, the handle 20 appears to have an elongated S-shape. Accordingly, the handle 20 can be divided into three segments, a lower segment 118, an upper segment 120 and a horizontal segment 122. At the junction between the lower segment 118 and the upper segment 120 is a finger rest 124. The finger rest 124 is dimensioned such that the index finger or thumb of a user's hand can comfortably rest therein. The lower segment 118 curves away from the housing 12. When the user holds the pump with her index finger in the finger rest 124, the rest of her fingers curve around the lower segment 118. Alternatively, her fingers can wrap around the body 16 of the breast pump 10 with her thumb in the finger rest 124. The upper segment 120 is, for example, shaped to follow the contour of the upper compartment 26 of the housing 12. Located on the underside of the upper segment 120 and the horizontal segment 122 are, for example, a plurality of structural ribs 126 that provide structural rigidity to the handle 20. Located on the underside of the horizontal segment 122 near the junction with the upper segment 120 are a pair of hooks 128. Located at the end of the upper segment 120 opposite from that of the hooks 128 are a pair of eyelets 130.

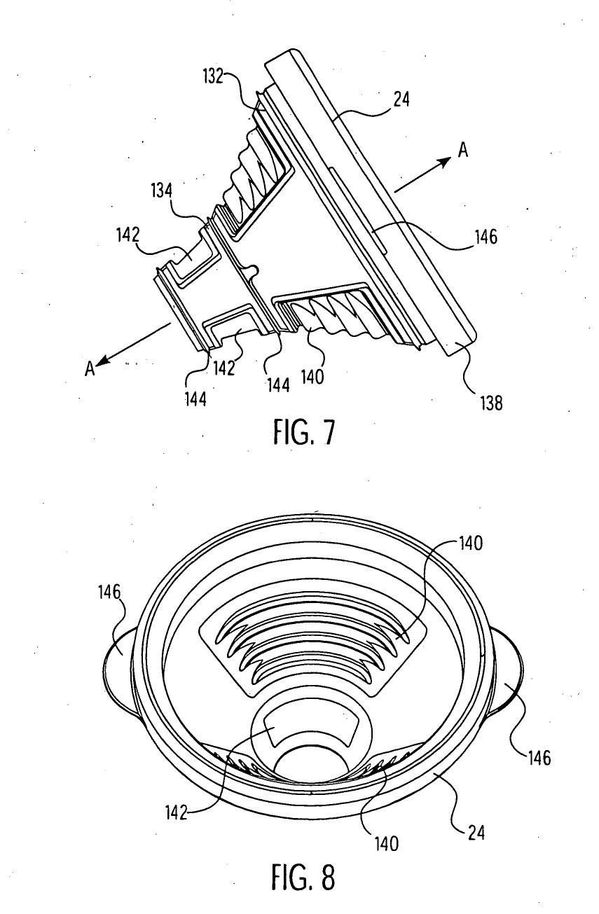

FIGS. 7 and 8 show a side elevation view and a front perspective view of the inner funnel 24 respectively. The inner funnel 24 sealingly engages the user's breast during use. The shape of the profile of the inner funnel 24, for example, has the appearance of a truncated cone. The inner funnel 24 comprises a proximal section 132 and a distal section 134. When the inner funnel 24 is placed on a breast, the proximal section 132 is, for example, physically located about the milk lake region of the breast, whereas the distal section 134 is, for example, physically located about the nipple.

The proximal section 132 includes at least one area in the form of a flexible proximal membrane 140. As used herein the term “flexible” means capable of being flexed or bended in response to positive pressure, negative pressure and changes thereof (i.e., resilient and reversibly deformable). The proximal membrane 140 is made of, for example, a thermoplastic elastomer, such as DYNAFLEX available from GLS Corporation (McHenry, Ill.). Although optional, it is desirable that the proximal membrane 140 be corrugated. FIG. 8 shows, for example, two proximal membranes 140 symmetrically positioned about the longitudinal axis A of the funnel portion 14. An alterative embodiment can have three flexible proximal membranes 140 evenly spaced along the proximal section 132. The remaining areas of the proximal section 132 frame and support the proximal membranes 140 and are made of a rigid material, for example, polypropylene.

Although optional, it is desirable to have at least one area of the distal section 134 to be a flexible distal membrane 142. Like the proximal membrane 140, the distal membrane 142 can be either smooth or corrugated and be made from a thermoplastic elastomer. Two distal membranes 142 are shown in FIG. 7. Similar to the proximal section 132 the remaining section of the distal section 134 frame and support the distal membranes 142.

The proximal membranes 140 and the distal membranes 142 are, for example, attached to the rigid sections of the proximal section 132 and the distal section 134 respectively by overmolding. Also overmolded onto the inner funnel 24 is at least one ring seal 144. As shown in FIG. 7, for example, two ring seals 144 are present, one located at the junction of the proximal section 132 and the distal section 134, and one other located at the distal end of the distal section 134. When the inner funnel 24 is placed within the outer funnel 22, the ring seals 144 sealingly contact the inside surface of the outer funnel 22.

Located on the proximal side of the proximal section 132 is an attachment ring 138 that allows the inner funnel 24 to be releasably attached to the outer funnel 22 by using, for example a tongue and groove interface. Extending from the attachment ring 138 are, for example, tabs 146 that allow the user to grasp the attachment ring 138 to separate the inner funnel 24 from the outer funnel 22.

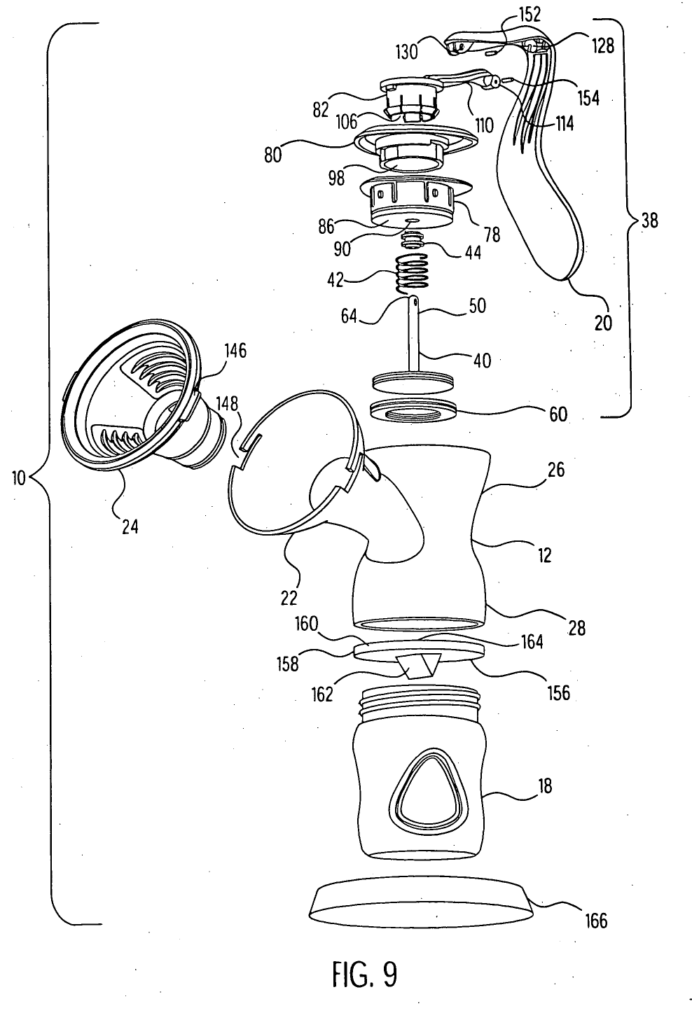

FIG. 9, shows an exploded perspective view of the entire breast pump 10. To assemble the breast pump 10, the inner funnel 24 is attached to the outer funnel 22. For example, funnel grooves 148 on the proximal circumference of the outer funnel 22 are dimensioned to releasably receive the tabs 146 of the inner funnel 24. Thus, the inner funnel 24, for example, form a snap-fit with the outer funnel 22. Alternatively, threading can be used on the inner funnel 24 and the outer funnel 22 such that the inner funnel 24 screws onto the outer funnel 22. Yet another possible embodiment is to use a tongue and groove fit, for example, a TUPPERWARE seal, that is the type of seal between the lid and container of commonly found TUPPERWARE brand storage containers sold by The Tupperware Corporation of Orlando, Fla. Such a TUPPERWARE seal comprises a groove or channel along the distal circumference of the larger rim of the inner funnel 24. The proximal circumference of the outer funnel 22 is releasably received within the groove of the inner funnel 24. Any other means of attaching the inner funnel 24 to the outer funnel 22 that provided that an airtight seal is formed between the inner funnel 24 and the outer funnel 22. Another alternative, is to have the inner funnel 24 and the outer funnel 22 welded, for example by ultrasonic welding or fused together of formed as an integral component.

In the actuator 38 sub-assembly, the spring 42 is placed on the rod 50 of piston 40 such that the rod 50 extends through the spring 42. The grommet seal 44 is placed on the rod 50. The grommet seal 44 is inserted within the receiving hole 90 of the bottom wall 86 of the lid underside 78. The lid base 80 is attached to the lid underside 78 with the rotating center 82 inserted within the central compartment 88 as discussed above. The central compartment 88 is in fluid communication with the central bore 106. The rod 50 extends through the central compartment 88 and central bore 106 such that end 64 of the rod 50 exits the top opening of the central bore 106 and thus exiting the lid.

The handle 20 rests on top of the handle support 110 such that the eyelets 130 of the handle rest within the bore 106 of the rotating center. The centers of the openings of the hooks 128 are axially aligned with the support receiving bore 114. A second fastening pin 154 is inserted through the support receiving bore 114 and the openings of the hooks 128. The second fastening pin 154 acts as the pivot point when the handle 120 is depressed against the housing 12. Once the handle 20 is assembled to the actuator 38, the combination of the handle 20 and actuator 38 subassembly can be attached to the upper compartment 26 of the housing 12 by turning the combination a quarter turn clockwise.

Located in the lower compartment 28 of the housing 12 is a valve element 156. The valve element 156, for example is a one-way duckbill valve that includes a fitting portion 158 and a duckbill portion 162. The fitting portion 158 comprises an annular ring 160 defining a flow-through 164 that leads through a pair of opposing flexible lips that flex apart to permit the flow of breast milk through the valve element 156 and into the bottle 18; the lips, however, collapse together to prevent the flow of breast milk in the opposite direction. The outer circumference of the annular ring 160 sealingly engages the inner surface of the lower compartment 28. The valve element 156 can be held within the lower compartment 28, for example, by a friction-fit.

As discussed before, a bottle 18 is releasably screwed into the lower compartment 28 of the housing 12. Optionally, the breast pump 10 is removably inserted into a bottle stand 166 that provides additionally stability when an empty, partially filled or filled bottle 18 is still connected to the breast pump 10.

Although the breast pump 10 can be disassembled into all of the aforementioned components, it is desirable that the actuator 38 and handle 20 be pre-assembled into a single subassembly. Thus, breast pump 10 can be divided into the following four components, for example, the inner funnel 24; the actuator 38/ handle 20 subassembly; the valve element 156 and the bottle 18. A user only has to clean, sterilize and assemble the four aforementioned components. Four components are significantly less than the total number of discrete parts that comprise the breast pump 10. The breast pump 10 and the components therein can be cleaned using ordinary dish detergent and rinsed with clean water. Alternatively, the breast pump 10 can be used in a dishwasher.

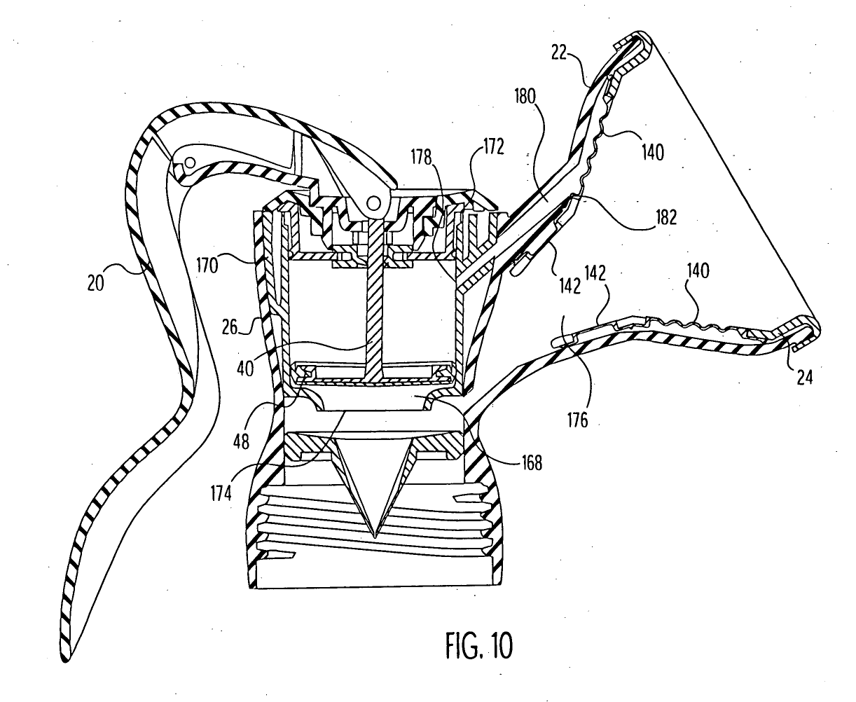

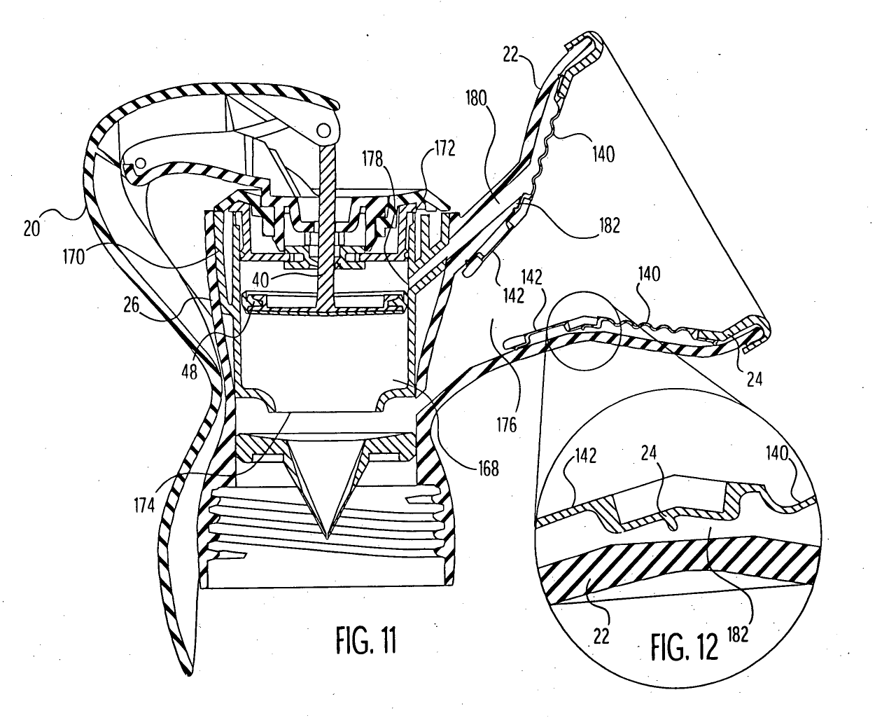

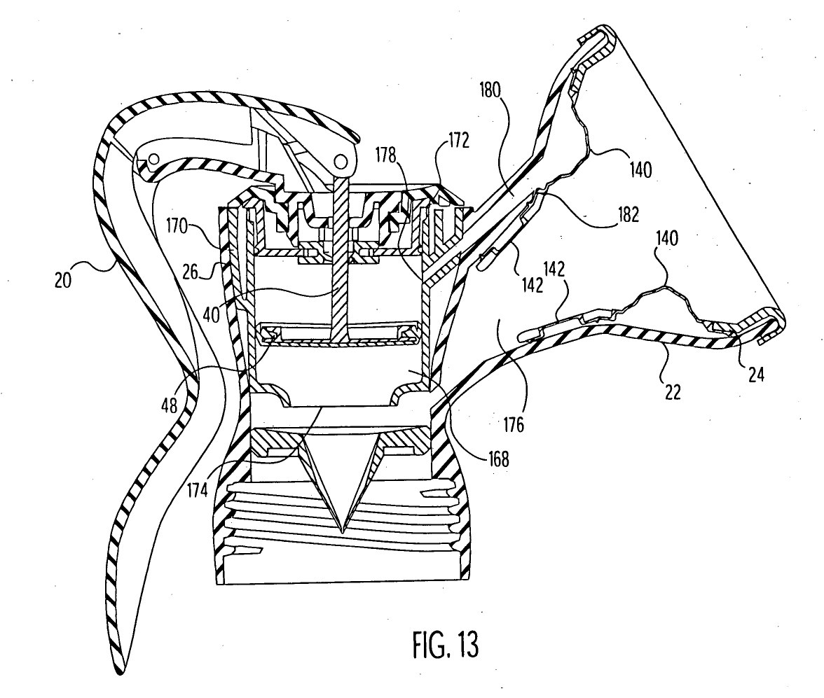

FIGS. 10, 11 and 13 show cross-sectional side views of an exemplary breast pump 10 without the bottle 18 connected thereto. The figures vary by the orientation of the handle 20 and the consequent change in position of the piston 40 relative to the housing 12. The phrase “neutral position” refers to the position of the handle 20 when no force, e.g., no squeezing, is being applied thereto by the user. The phrase “engaged position” refers to the position of the handle 20 when the handle 20 is squeezed and pressed completely against the housing 12 of the breast pump 10. Since the handle 20 is mechanically connected to the piston 40, as the handle 20 travels so too does the piston 40. The piston 40 travels cyclically in a vertical fashion within the chamber 168. Although, a cylindrical shaped chamber 168 is shown in the figures, it is contemplated that any volumetric shape can be used for the chamber 168. When the handle 20 is in the neutral position, the disc 48 of the piston 40 is in its lowest position, or as used herein the “starting point.” When the handle 20 is squeezed or depressed, the piston 40 moves upward within the chamber 168 until it reaches the “ending point.” As used herein the term “upstroke” refers to the travel of the piston 40 from the starting point to the ending point. Conversely, the piston 40 moves downward within the chamber 168 as the force applied to the handle 20 is diminished or released. As used herein the term “downstroke” refers to the travel of the piston 40 from the ending point to the starting point. As used herein, the term “stroke” refers to either a single upstroke or a single downstroke of the pressure generating mechanism. It is this cyclical movement of upstrokes and downstrokes that provide the positive and negative pressures conveyed to the breast within the funnel portion 14. During the upstroke of the piston 40, the pressure within the funnel portion changes from negative pressure to positive pressure. The “transition point” refers to the point during the upstroke in which the pressure within the funnel changes from negative pressure to positive pressure. FIGS. 10 and 11 show the handle 20 in its neutral position and engaged position respectively. FIG. 13 shows the handle 20 in a position in between the neutral position and engaged position such that the proximal membranes 140 are inflated but the distal membranes 142 have yet to be inflated (described in more detail below).

As shown in FIGS. 10, 11 and 13, cross-sections of the breast pump 10 are shown. Within the upper compartment 26 is a piston shell 170 that has an inner wall 172 that defines the chamber 168. The bottom perimeter of the chamber 168 defines a vacuum port 174. The vacuum port 174 is in fluid communication with the funnel interior 176 as defined by the inner funnel 24. Located within the inner wall 172 is a pressure port 178 that is in fluid communication to a pressure conduit 180. The pressure conduit 180 is defined by the space between the outer funnel 22 and the proximal membrane 140. In fluid communication with the pressure conduit 180 and defined by the inner funnel 22 and the outer funnel 24 is an inflation conduit 182 (shown in greater detail in FIG. 12). The inflation conduit 182 is also in fluid communication between the proximal membranes 140 and the distal membranes 142.

In operation, as the piston travels up from the starting point, negative pressure (e.g. a vacuum) is created and conveyed through the vacuum port 174 and into the inner funnel 24. Such a vacuum draws the mother's breast into the inner funnel 24 creating a seal. The distal membrane 142 and proximal membrane 140, for example, are aligned with the areola and the milk lake region respectively. Simultaneously with the upstroke of the piston, air is displaced from the chamber 168 and flows into the pressure conduit 180. The movement of air into the pressure conduit 180 creates positive pressure that displaces the proximal membrane 140. As used herein the term “inflation” refers to the displacement of a membrane (either a proximal membrane 140 or distal membrane 142) due to the application of a positive pressure. This displacement of the proximal membrane 140 applies positive pressure to the milk lake region thereby massaging the milk lake region. Because the pressure conduit 180 is connected to the inflation conduit 182, some of the air bleeds from the pressure conduit 180 and into the inflation conduit 182 causing the distal membrane 142 to inflate. The displacement of the distal membrane 142 applies positive pressure to the areola region thereby massing the areola region. The rate that the distal membrane 142 displaces is, for example, a function of the area of the opening connecting the pressure conduit 182 to the inflation conduit 182. A larger opening results in a faster displacement of the distal membrane 142, such that both the distal membrane 142 and the proximal membrane 140 are displacing at the same rate. By changing the volumes of either the pressure conduit 180 and the inflation conduit 182 or changing the area of the opening, the rate and/or sequence of the inflation, and consequently massage, can be adjusted. For example, the proximal membrane 140 and distal membrane 142 can be designed such that they inflate simultaneously. Alternatively, they can be configured to inflate sequentially, for example, with the proximal membrane 140 inflating first. During the downstroke of the piston 40, the air that filled the membranes is transferred back to the chamber 168. Once the piston 40 returns back to the starting point, both the positive and negative pressure conveyed to the funnel interior 176 ends.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

1. A breast pump comprising:
a housing having a funnel portion, said funnel portion comprising an outer funnel and an inner funnel received in said outer funnel and said inner funnel defining a funnel interior adapted to sealingly engage a breast;
at least one flexible membrane formed in the inner funnel;
a chamber defined by an inside wall of said housing, said chamber adapted to receive a pressure generating mechanism and having a vacuum port in fluid communication with said funnel interior and having a pressure port;
a pressure conduit in fluid communication with said pressure port and said membrane;
wherein during a stroke of said pressure generating mechanism a negative pressure is conveyed to said funnel interior and a positive pressure is conveyed to via said pressure conduit to displace said flexible membrane.

2. The breast pump of claim 1, wherein said pressure generating mechanism is a piston.

3. The breast pump of claim 1, wherein said flexible membrane contacts a milk lake region of said breast when displaced.

4. The breast pump of claim 1, wherein said flexible membrane contacts an areola of said breast when displaced.

5. The breast pump of claim 1, wherein said flexible membrane is corrugated.

6. The breast pump of claim 1, wherein said negative pressure and positive pressure are generated simultaneously during said stroke of said pressure generating mechanism.

7. The breast pump of claim 1, further comprising a handle connected to said pressure generating mechanism, wherein movement of said handle corresponds to movement of said pressure generating mechanism within said chamber.

8. The breast pump of claim 1, wherein said breast pump is a manual breast pump.

9. A manual breast pump comprising:
a housing having a funnel portion, said funnel portion comprising an outer funnel and an inner funnel received in said outer funnel and said inner funnel defining a funnel interior adapted to sealingly engage a breast;
a first flexible membrane formed in the inner funnel;
a second flexible membrane formed in the inner funnel, said second flexible membrane in fluid communication with said first flexible membrane via an inflation channel;
a chamber defined by an inside wall of said housing, said chamber adapted to receive a pressure generating mechanism and having a vacuum port in fluid communication with said funnel interior and having a pressure port;
a pressure conduit in fluid communication with said pressure port and said first flexible membrane;
wherein during a stroke of said pressure generating mechanism a negative pressure is conveyed to said funnel interior and a positive pressure is conveyed to via said pressure conduit to displace said first flexible membrane and said second flexible membrane.

10. The breast pump of claim 9, wherein said pressure generating mechanism is a piston.

11. The breast pump of claim 9, wherein said first flexible membrane contacts a milk lake region of said breast when displaced and said second flexible membrane contacts a nipple of said breast when displaced.

12. The breast pump of claim 9, wherein said first flexible membrane is corrugated.

13. The breast pump of claim 11, wherein said negative pressure and positive pressure are generated simultaneously during said stroke of said pressure generating mechanism.

14. The breast pump of claim 9, wherein said first flexible membrane begins displacement prior to a displacement of said second flexible membrane.
